(19) United States
(12) Patent Application Publication
(10) Pub. No.: US 2022/0287579 A1
(43) Pub. Date: Sep. 15, 2022

(54) SYSTEM AND METHOD FOR CONTINUOUS NON-INVASIVE BLOOD PRESSURE MEASUREMENT

(71) Applicant: Covidien LP, Mansfield, MA (US)

(72) Inventors: Paul S. Addison, Scotland (GB); Dean Montgomery, Edinburgh (GB); Andre S.M. Antunes, Edinburgh (GB)

(21) Appl. No.: 17/201,437

(22) Filed: Mar. 15, 2021

(51) Int. Cl. A61B 5/021; A61B 5/00

(52) U.S. Cl. CPC A61B 5/02125; A61B 5/7264; A61B 5/02141; A61B 5/02116; A61B 2560/0223

(57) ABSTRACT

The present technology relates to patient monitoring systems and methods using plural PPG sensors in contact with a patient at different locations, wherein a comparison of the PPG data is performed to calculate a differential pulse transit time (DPTT) between the first and second locations, followed by a determination of continuous non-invasive blood pressure (CNIBP) using the PPG data from the plural and the calculated DPTT.

### FIELD

The present technology is generally related to a system and method for continuous non-invasive blood pressure (CNIBP) measurement, for example using deep learning artificial intelligence (AI) utilizing differential pulse transit time (DPTT).

### BACKGROUND

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient uses attenuation of light to determine physiological characteristics of a patient. This is used in pulse oximetry, and the devices built are based upon pulse oximetry techniques. Light attenuation is also used for regional or cerebral oximetry. Oximetry may be used to measure various blood characteristics, such as the oxygen saturation of hemoglobin in blood or tissue, the volume of individual blood pulsations supplying the tissue, and/or the rate of blood pulsations corresponding to each heartbeat of a patient. The signals can lead to further physiological measurements, such as respiration rate, glucose levels or blood pressure.

Many conventional medical monitors require attachment of a sensor to a patient in order to detect physiologic signals from the patient and to transmit detected signals through a cable to the monitor. These monitors process the received signals and determine vital signs such as the patient's pulse rate, respiration rate, and arterial oxygen saturation. For example, a pulse oximeter is a finger sensor that can include two light emitters and a photodetector. The sensor emits light into the patient's finger and transmits the detected light signal to a monitor. The monitor includes a processor that processes the signal, determines vital signs (e.g., pulse rate, respiration rate, arterial oxygen saturation), and displays the vital signs on a display.

Other monitoring systems include other types of monitors and sensors, such as electroencephalogram (EEG) sensors, blood pressure cuffs, temperature probes, air flow measurement devices (e.g., spirometer), and others. Some wireless, wearable sensors have been developed, such as wireless EEG patches and wireless pulse oximetry sensors.

Determination of blood pressure non-invasively and continuously presents a significant technical challenge in the medical device industry. For that reason, blood pressure is typically measured intermittently via a separate blood pressure cuff or continuously using invasive techniques, for example using of an invasive arterial line, with the various monitoring devices being connected to one or more patient monitors to present patient measurements.

What is needed in the art are systems and methods allowing for continuous, non-invasive blood pressure measurement.

### SUMMARY

The techniques of this disclosure generally relate to systems and methods for continuous non-invasive blood pressure (CNIBP) measurement. In exemplary aspects described herein, CNIBP is measured using deep learning artificial intelligence (AI) utilizing differential pulse transit time (DPTT).

In one aspect, a patient monitoring system includes a first PPG sensor in contact with a patient at a first location, the first sensor providing first data over a first time period related to the patient to determine one or more patient parameters; a second PPG sensor in contact with a patient at a second patient location different from the first location, the second sensor providing second data over said first time period related to the patient to determine one or more patient parameters; and a processor configured to: compare at least a portion of the first data and at least a portion of the second data in order to calculate a differential pulse transit time (DPTT) between the first and second locations; and determine continuous non-invasive blood pressure (CNIBP) using the first data, the second data and the DPTT.

In another aspect, a method for patient monitoring includes configuring a first PPG sensor to contact a patient at a first location, the first sensor configured to provide first data over a first time period related to the patient to determine one or more patient parameters; configuring a second PPG sensor to contact a patient at a second patient location different from the first location, the second sensor providing second data over said first time period related to the patient to determine one or more patient parameters; and with a processor: comparing at least a portion of the first data and at least a portion of the second data in order to calculate a differential pulse transit time (DPTT) between the first and second locations; and determining continuous non-invasive blood pressure (CNIBP) using the first data, the second data and the DPTT.

In an exemplary aspect, the processor compares at least one fiducial point in the first data and in the second data in order to calculate DPTT. Exemplary fiducial points include: a peak of the pulse, the trough of the pulse; or the location of maximum upslope gradient.

In another exemplary aspect, at least a portion of the first sensor data, at least a portion of the second sensor data, and the calculated DPTT are input into a deep learning AI model to determine CNIBP. Exemplary deep learning AI models include an LSTM model, a CNN model, and a hybrid CNN-LSTM model.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a schematic view of a patient monitoring system configured with plural pulse oximetry sensors, configured in accordance with various embodiments of the present technology;

FIG. 2 is a schematic view of the monitor and one sensor of patient monitoring system of FIG. 1, configured in accordance with various embodiments of the present technology;

FIG. 3 is a comparative chart view of exemplary blood pressure and PPG signals, in accordance with various embodiments of the present technology;

FIG. 4 is another comparative chart view of exemplary blood pressure and PPG signals, in accordance with various embodiments of the present technology;

FIG. 5 is a chart view illustrating calculation of DPTT relative to PPG signals of two sensors;

FIG. 6 is a block view of deep learning AI inputs, in accordance with various embodiments of the present technology;

FIG. 7 is another block view of deep learning AI inputs, in accordance with various embodiments of the present technology;

FIG. 8 is a flow chart of an exemplary LSTM machine learning model;

FIG. 9 is a flow chart of an exemplary method using an LSTM model, in accordance with various embodiments of the present technology;

FIG. 10 is a flow chart of an exemplary CNN machine learning model;

FIG. 11 is a flow chart of an exemplary method using a CNN model, in accordance with various embodiments of the present technology;

FIG. 12 is a flow chart of an exemplary method using a hybrid CNN-LSTM model, in accordance with various embodiments of the present technology; and

FIG. 13 is a chart view of an output for an LSTM model, in accordance with various embodiments of the present technology.

### DETAILED DESCRIPTION

The following disclosure describes systems and methods for continuous non-invasive blood pressure (CNIBP) measurement. In exemplary aspects described herein, CNIBP is measured using deep learning artificial intelligence (AI) utilizing differential pulse transit time (DPTT).

In exemplary aspects, devices, systems, and/or methods configured in accordance with embodiments of the present technology can include one or more sensors or probes associated with (e.g., contacting) a patient that can be configured to capture data (e.g., temperature, blood pressure, heart rate, arterial oxygen saturation, etc.) related to a patient. The devices, systems, and/or methods can transmit the captured data to a monitoring device, hub, mobile patient management system (MPM), or the like. In some embodiments, the devices, systems, and/or methods can analyze the captured data to determine and/or monitor one or more patient parameters. In these and still other embodiments, the devices, systems, and/or methods can trigger alerts and/or alarms when the devices, systems, and/or methods detect one or more patient parameter abnormalities.

In some embodiments, one or more sensors or probes associated with (e.g., contacting) a patient can be configured to capture data related to a patient, e.g. as a PPG signal. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The amount of light detected or absorbed may then be used to calculate any of a number of physiological parameters, including oxygen saturation (the saturation of oxygen in pulsatile blood, SpO2), an amount of a blood constituent (e.g., oxyhemoglobin), as well as a physiological rate (e.g., pulse rate or respiration rate) and when each individual pulse or breath occurs. For SpO2, red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, and/or from curve fitting and/or other interpolative techniques.

In exemplary aspects described herein, CNIBP is measured using deep learning artificial intelligence (AI) utilizing differential pulse transit time (DPTT) utilizing plural PPG signals obtained via separate locations of a patient. The DPTT may be defined as the difference in time that a pulse wave takes to arrive at two distinct arterial locations. The DPTT derived from the PPG signals acquired from pulse oximeter probes placed at such distinct locations may be used as an input in an AI model to determine CNIBP.

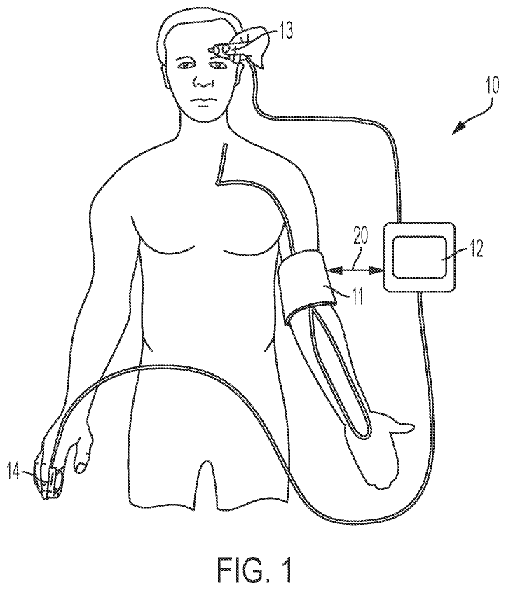

Specific details of several embodiments of the present technology are described herein with reference to FIGS. 1-12. Although many of the embodiments are described with respect to devices, systems, and methods for CNIBP monitoring of a human patient, other applications and other embodiments in addition to those described herein are within the scope of the present technology. For example, at least some embodiments of the present technology can be useful for detection and/or monitoring of one or more parameters of other animals and/or in non-patients (e.g., elderly or neonatal individuals within their homes, individuals in a search and rescue or stranded context, etc.). It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

FIG. 1 is a schematic view of an exemplary monitoring system shown generally at 10, including a patient monitor 12 and sensors 13 and 14, such as a pulse oximetry sensor, to monitor physiological parameters of a patient. By way of example, the sensors may be a NELLCOR™, or INVOS™ sensor available from Medtronic (Boulder, Colo.), or another type of oximetry sensor. Sensor 14 is provided on a patient's fingertip; and sensor 13 is provided on the patient's forehead. Although some depicted embodiments relate to sensors for use on a patient's fingertip and forehead, it should be understood that, in certain embodiments, the features of the sensors as provided herein may be incorporated into sensors for use on other tissue locations (e.g., toe, wrist, earlobe, etc.). Further, exemplary systems may include more than two sensors provided at discrete arterial locations of a patient. Referring still to FIG. 1, an optional blood pressure cuff is shown at 11. The blood pressure cuff includes a communications link 20, which may be wired or wireless.

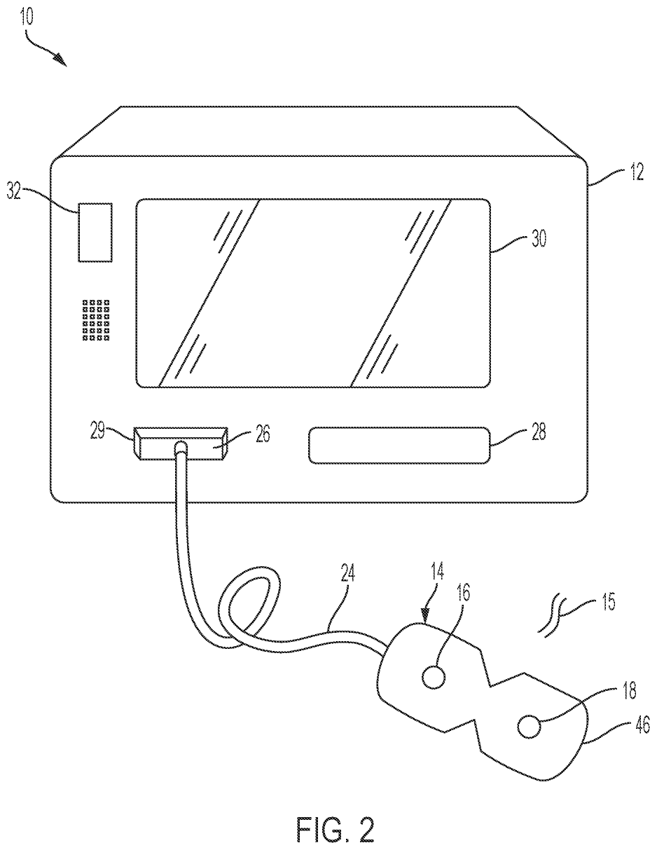

FIG. 2 is a schematic view showing the exemplary patient monitor 12 and sensor 14 in more detail. In the embodiment of FIG. 2, the sensor 14 is a pulse oximetry sensor that includes one or more emitters 16 and one or more detectors 18. For pulse oximetry applications, the emitter 16 transmits at least two wavelengths of light (e.g., red and infrared (IR)) into a tissue of the patient. For other applications, the emitter 16 may transmit 3, 4, or 5 or more wavelengths of light into the tissue of a patient. The detector(s) 18 include a photodetector selected to receive light in the range of wavelengths emitted from the emitter(s) 16, after the light has passed through the tissue. Additionally, the emitter(s) 16 and the detector(s) 18 may operate in various modes (e.g., reflectance or transmission).

The sensor 14 also includes a sensor body 46 to house or carry the components of the sensor 14. The body 46 includes a backing, or liner, provided around the emitter 16 and the detector 18, as well as an adhesive layer (not shown) on the patient side. The sensor 14 may be reusable (such as a durable plastic clip sensor), disposable (such as an adhesive sensor including a bandage/liner), or partially reusable and partially disposable.

In the embodiments shown, the sensors 14 is communicatively coupled to the patient monitor 12. In certain embodiments, the sensors may include a wireless module configured to establish a wireless communication 15 with the patient monitor 12 using any suitable wireless standard. For example, the sensors may include a transceiver that enables wireless signals to be transmitted to and received from an external device (e.g., the patient monitor 12, a charging device, etc.). The transceiver may establish wireless communication 15 with a transceiver of the patient monitor 12 using any suitable protocol. For example, the transceiver may be configured to transmit signals using one or more of the ZigBee standard, 802.15.4x standards WirelessHART standard, Bluetooth standard, IEEE 802.11x standards, or MiWi standard. Additionally, the transceiver may transmit a raw digitized detector signal, a processed digitized detector signal, and/or a calculated physiological parameter, as well as any data that may be stored in the sensor, such as data relating to wavelengths of the emitters 16, or data relating to input specification for the emitters 16. Additionally, or alternatively, the emitters 16 and detectors 18 of the sensor 14 may be coupled to the patient monitor 12 via a cable 24 through a plug 26 (e.g., a connector having one or more conductors) coupled to a sensor port 29 of the monitor. In certain embodiments, the sensor 14 is configured to operate in both a wireless mode and a wired mode. Accordingly, in certain embodiments, the cable 24 is removably attached to the sensor 14 such that the sensor 14 can be detached from the cable to increase the patient's range of motion while wearing the sensor 14. It should be recognized that wired or wireless configurations, as with sensor 14, are also contemplated with regard to sensor 13, as well as optional blood pressure cuff 11, which are shown in FIG. 1.

The patient monitor 12 is configured to calculate physiological parameters of the patient relating to the physiological signal received from the sensors 13, 14. For example, the patient monitor 12 may include a processor configured to calculate the patient's arterial blood oxygen saturation, tissue oxygen saturation, pulse rate, respiration rate, blood pressure, blood pressure characteristic measure, autoregulation status, brain activity, and/or any other suitable physiological characteristics. Additionally, the patient monitor 12 may include a monitor display 30 configured to display information regarding the physiological parameters, information about the system (e.g., instructions for disinfecting and/or charging the sensor 14), and/or alarm indications. The patient monitor 12 may include various input components 32, such as knobs, switches, keys and keypads, buttons, etc., to provide for operation and configuration of the patient monitor 12. The patient monitor 12 may also display information related to alarms, monitor settings, and/or signal quality via one or more indicator lights and/or one or more speakers or audible indicators. The patient monitor 12 may also include an upgrade slot 28, in which additional modules can be inserted so that the patient monitor 12 can measure and display additional physiological parameters.

Because the sensors 13, 14 may be configured to operate in a wireless mode and, in certain embodiments, may not receive power from the patient monitor 12 while operating in the wireless mode, the sensors 13, 14 may include a battery to provide power to the components of the sensor (e.g., the emitter(s) 16 and the detector(s) 18). In certain embodiments, the battery may be a rechargeable battery such as, for example, a lithium ion, lithium polymer, nickel-metal hydride, or nickel-cadmium battery. However, any suitable power source may be utilized, such as, one or more capacitors and/or an energy harvesting power supply (e.g., a motion generated energy harvesting device, thermoelectric generated energy harvesting device, or similar devices).

As noted above, in an embodiment, the patient monitor 12 is a pulse oximetry monitor and the sensor 14 is a pulse oximetry sensor. The sensor 14 may be placed at a site on a patient with pulsatile arterial flow, typically a fingertip, toe, forehead or earlobe, or in the case of a neonate, across a foot. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. As shown in FIG. 1, the patient monitoring system 10 may include sensors 13, 14 at multiple locations. The emitter 16 emits light which passes through the blood perfused tissue, and the detector 18 photoelectrically senses the amount of light reflected or transmitted by the tissue. The patient monitoring system 10 measures the intensity of light that is received at the detector 18 as a function of time.

As we have noted, exemplary systems and methods described herein determine continuous non-invasive blood pressure (CNIBP) using an AI model, where the inputs include, among other possible inputs, differential pulse transit time (DPTT). Referring to FIG. 1, PPG signals from pulse oximeter sensors 13, 14, which are placed at separate patient locations may be used to derive DPTT, which may be defined as the difference in time that a pulse wave takes to arrive at two distinct arterial locations.

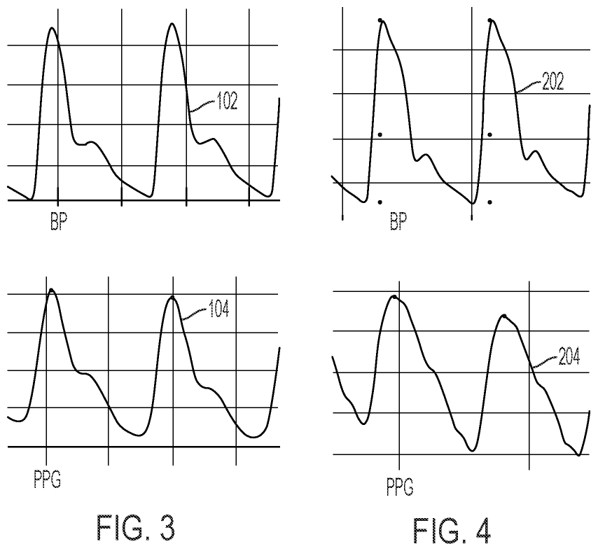

FIG. 3 shows a blood pressure (BP) signal 102 and a PPG signal 104 taken at around the same time. As can be seen, signals 102 and 104 look relatively similar in morphology.

However, FIG. 4 shows two different signals, 202 and 204, also taken at similar times, but with different looking morphology. In FIG. 4, the blood pressure (BP) signal 202 is taken using an (invasive) arterial line, whereas the PPG signal is generated through the volume change at the finger, generated as the arterial pulse (shock wave) hits the peripheries and is measured by a pulse oximeter. Mapping this pressure-mechanical-light coupling, from BP to PPG (or vice-versa) is difficult to achieve due to numerous confounders, including influence of contact force, ambient temperature, drugs, vasomotion, movement, arterial stiffening, posture changes, etc. The present disclosure recognizes these challenges and presents a solution by examining PPG signals at two separate points on the body, e.g., at the finger and forehead as in FIG. 1, to determine differential pulse transit time (DPTT). In doing so, systems and methods described herein advantageously avoid the problems associated traditional pulse transit time (PTT) taken between an electrocardiogram (ECG) signal and the pulse wave, which includes electromechanical delay (EMD).

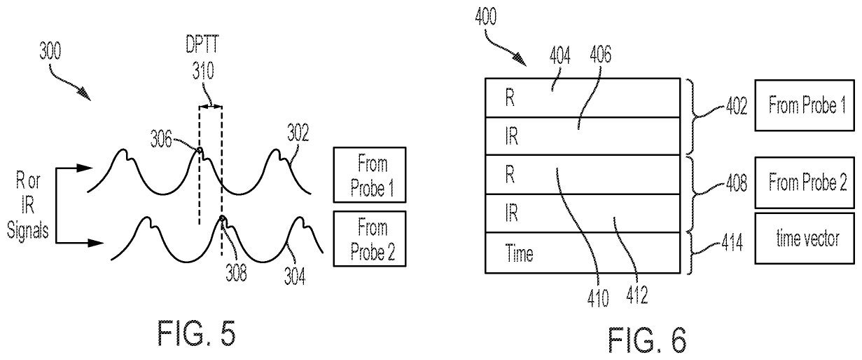

In exemplary embodiments, and with further reference to FIG. 1, DPTT is calculated from two PPG signals taken at different body locations by measuring the time difference between two corresponding fiducial points on each signal. FIG. 5 illustrates a first PPG signal from a first pulse oximetry sensor/probe (e.g., sensor 13 in FIG. 1) at 302 and a second PPG signal from a second pulse oximetry sensor (e.g., sensor 14 in FIG. 1) at 304. The PPG signals used may be from the same or different wavelengths of light, e.g., red signals from sensors 13, 14 in FIG. 1, infrared signals from sensors 13, 14, a red signal from sensor 13 and an infrared signal from sensor 14, or an infrared signal from sensor 13 and a red signal from sensor 14

FIG. 5 illustrates generally at 300 PPG data from two pulse oximetry sensors at different locations on the body. With regard to FIG. 5, one or more corresponding fiducial points on each signal may be identified, for example the peak of the pulses in each PPG signal, in order to measure the time difference between those fiducial points for the DPTT calculation. Peak 306 is identified on signal 302. Peak 308 is identified on signal 304. DPTT is shown at 310 as the difference in time between fiducial points 306 and 308. It should be noted that while peaks 306 and 308 are shown in FIG. 5, other fiducial points are contemplated herein, for example, the trough, the location of maximum upslope gradient, or other corresponding points. Further, any two locations on the body may be used to produce a DPTT, e.g., finger and toe, ear and toe, finger and forehead, etc.

In further exemplary embodiments, systems and methods described herein make use of both wavelengths from two or more PPG signals in a deep learning model. For example, FIG. 6 illustrates deep learning model inputs generally at 400, including red 404 and infrared 406 PPG signals from a first pulse oximetry sensor 402, red 410 and infrared 412 PPG signals from a second pulse oximetry sensor 408 and a time vector 414 for the PPGs. In such a way, the system and method can capture DPTT information, as well as morphological information at each location that is contained within each PPG and correlate to BP changes.

While FIG. 6 illustrates the use of two sets of PPG signals, from two locations, as inputs to a deep learning method, we note that any number of sets of signals from different sites may be utilized. For example, use of pulse oximetry sensors at the finger, toe and forehead would allow for the generation of three sets of DPTTs corresponding to finger-forehead, forehead-toe and toe-finger.

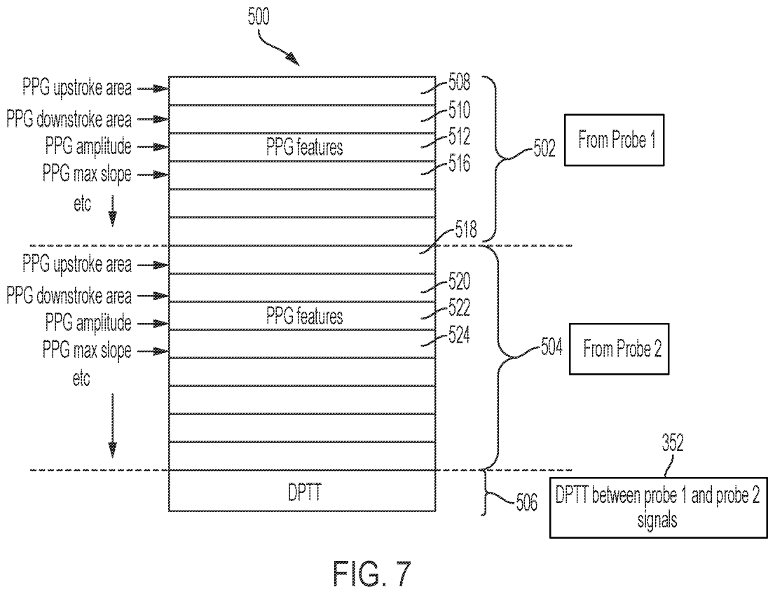

In other exemplary embodiments, inputs may be provided corresponding to features derived from the raw PPG signals. For example, these may include characteristic features from each PPG waveform including: pulse duration, relative position of maximum upslope of the systolic rise, peak location and amplitude, perfusion index, baseline trend, respiratory cycle information, area of upstroke, downstroke, max gradient of upslope, baseline value, etc. Additionally, for each feature a sequence of values over time may be used. These may be once per period of time (i.e. once per second, or once per pulse, or a single value from a time window of longer period (e.g. 15 seconds, 30 seconds, etc.) In addition to these features, the DPTT calculated between each signal may be provided as an input. Thus, a matrix of feature values from each signal and the corresponding DPTT may be constructed, as shown generally at 500 in FIG. 7.

FIG. 7 shows a first matrix of features from a first pulse oximetry probe at 502; a second matrix of features from a second pulse oximetry probe at 504; and DPTT between the location of the first probe and the second probe. The matrix of features for each probe may be selected, e.g., from features described above. For example, the matrix 502 from the first probe may include PPG upstroke area 508, PPG downstroke area 510, PPG amplitude 512, PPG maximum slope 516, etc. Matrix 504 includes the corresponding features from the second probe, with PPG upstroke area 518, PPG downstroke area 520, PPG amplitude 522, PPG maximum slope 524, etc.

Table 1, below outlines further exemplary features, for example relative to a finger PPG and the DPTT between the finger and a forehead:

TABLE 1

| Feature Name | Feature Definition |
|---|---|
| amplitude_respiration | amplitude of respiration component in the PPG |
| IR_pulse_duration | IR PPG pulse duration |
| IR_sys_relative_position | Position of the systolic peak in the pulse (percentage). If systolic |
| | peak happens half-way between two diastoles, value is 50 |
| IR_maxslope_relative_position | Position of the max slop (percentage in the pulse), same units as |
| | IR_sys_relative_position |
| IR_pulse_amplitude | IR PPG pulse amplitude |
| IR_pulse_amplitude_over_DC | IR PPG pulse amplitude normalised by signal baseline (DC) |
| IR_trend | how much the pulse rises/drops (difference between two diastolic |
| | points) - (in units of PPG/DC) |
| IR_trend_pct | how much the pulse rises/drops (as a percentage of the pulse |
| | amplitude) |
| IR_mean | mean of the PPG over the pulse (integral divided by duration) |
| IR_mean_over_DC | IR_mean divided by the DC value |
| IR_max_gradient_upstroke | maximum gradient of the initial upslope of the PPG pulse |
| IR_perfusion_index | perfusion index of the IR PPG over the pulse |
| IR_DC | mean baseline value of the IR PPG over the pulse |
| IR_area_upstroke | Area of the pulse from the initial diastolic until the systolic |
| IR_area_upstroke_over_DC | IR_area_upstroke divided by DC |
| IR_area_downstroke | Area of the pulse from the systolic peak until the final diastolic |
| IR_area_downstroke_over_DC | IR_area_downstroke divided by DC |
| IR_width_50 | Pulse width, measured at height = 50% of pulse amplitude |
| IR_width_75 | Pulse width, measured at height = 75% of pulse amplitude |
| IR_width_90 | Pulse width, measured at height = 90% of pulse amplitude |
| IR_uphill_average_slope | Slope of the line defined by the points initial_diastolic and systolic |
| IR_downhill_average_slope | Slope of the line defined by the points systolic and final_diastolic |
| IR_skewness | skew of the pulse component |
| IR_kurtosis | kurtosis of the pulse component |
| DPTT | differential pulse transit time between finger and ear |

In exemplary embodiments described herein, the AI model is trained to calculate a blood pressure signal from the provided inputs. This may be any characteristic blood pressure including, for example, the systolic pressure (SP), diastolic pressure (DP), mean arterial pressure (MAP) or pulse pressure (PP).

In further exemplary embodiments, feature matrices are input into the training cycle of a deep learning model with a target BP value associated with it. The model is then trained to associate the PPG morphological feature sequences with the blood pressure values. The model may then be tested using a test set of feature sequences previously unseen by the model to estimate the associated blood pressure. In this way a model may be generated with a given performance in terms of associating the PPG-based input to a BP.

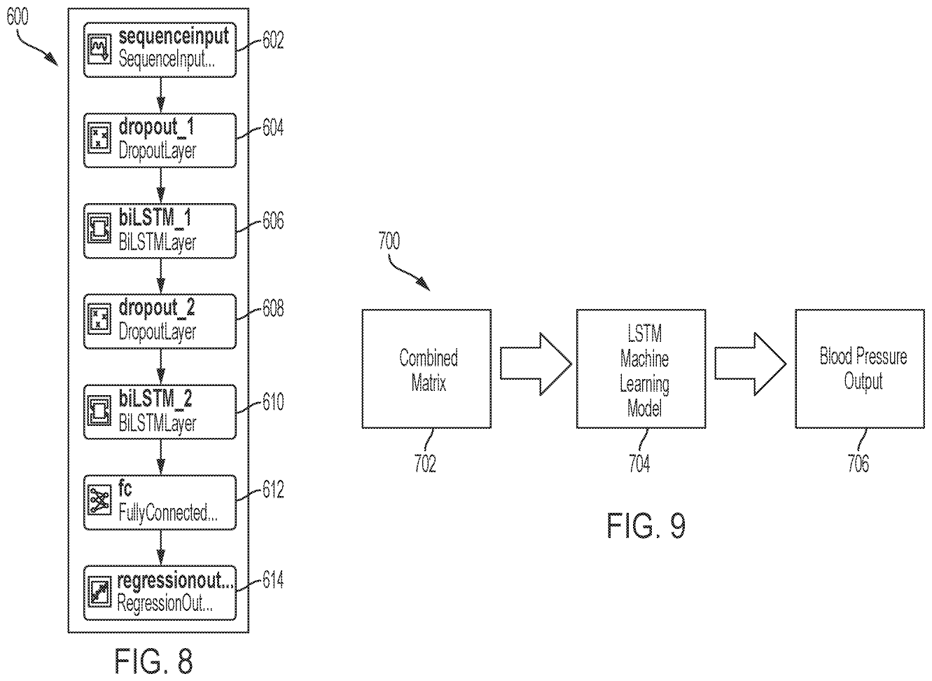

In exemplary embodiments described herein, the deep learning model is a long short-term memory (LSTM) machine learning model, with an exemplary architecture illustrated generally at 600 in FIG. 8. The exemplary architecture includes: an input layer (Sequence input block 602); a first dropout layer (DropoutLayer block 604); a first bidirectional LSTM layer (BiLSTMLayer block 606); a second dropout layer (DropoutLayer block 608); a second bidirectional LSTM layer (BiLSTMLayer block 610); a fully connected layer (FullyConnected block 612); and a regression output layer (RegressionOut block 614).

FIG. 9 provides a flowchart at 700, illustrating the features provided as a combined matrix 702 input into an LSTM machine learning model at 704, with a blood pressure output at 706.

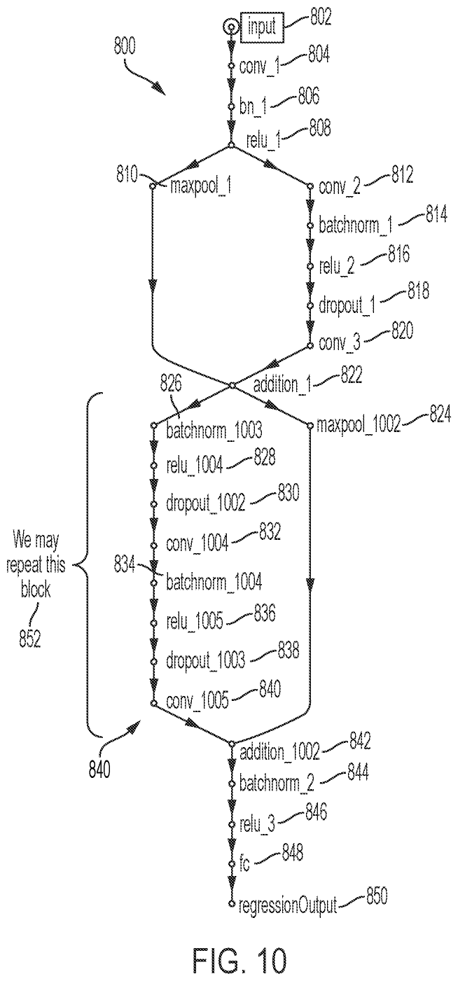

In further exemplary embodiments, the deep learning model is a convolutional neural network (CNN) model, an exemplary architecture of which is shown generally at 800 in FIG. 10. The exemplary architecture includes the following steps in the illustrated arrangement: input 802; convolution 804; batch normalization 806; ReLU (rectified linear unit) 808; max pooling 810; convolution 812; batch normalization 814; ReLU 816; dropout 818; convolution 820; addition 822; max pooling 824; batch normalization 826; ReLU 828; dropout 830; convolution 832; batch normalization 834; ReLU 836; dropout 838; convolution 840; addition 842; batch normalization 844; ReLU 846; fully connected layer 848; and regression output 850. We note that in exemplary embodiments the block indicated at 852 may be repeated during the process.

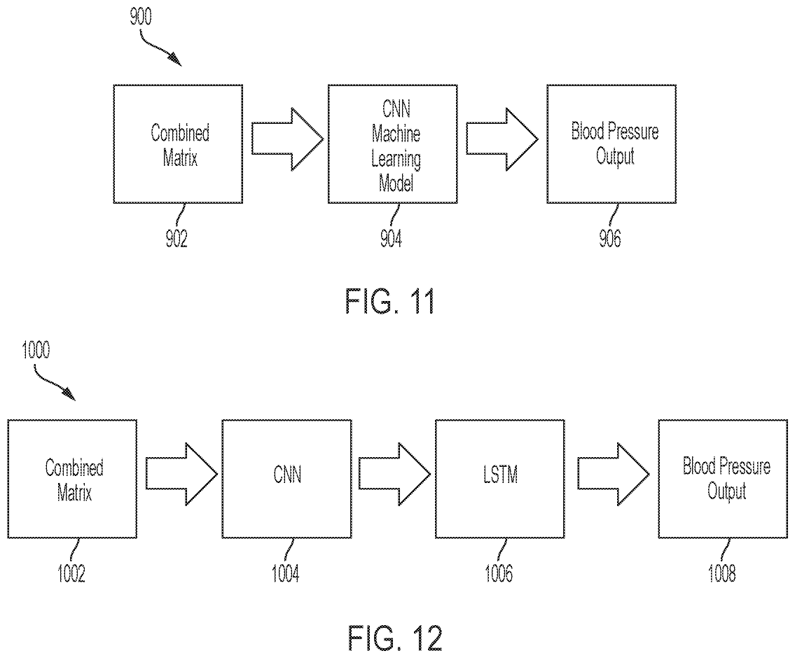

FIG. 11 provides a flowchart at 900, illustrating the features provided as a combined matrix 902 input into a CNN machine learning model at 904, with a blood pressure output at 906.

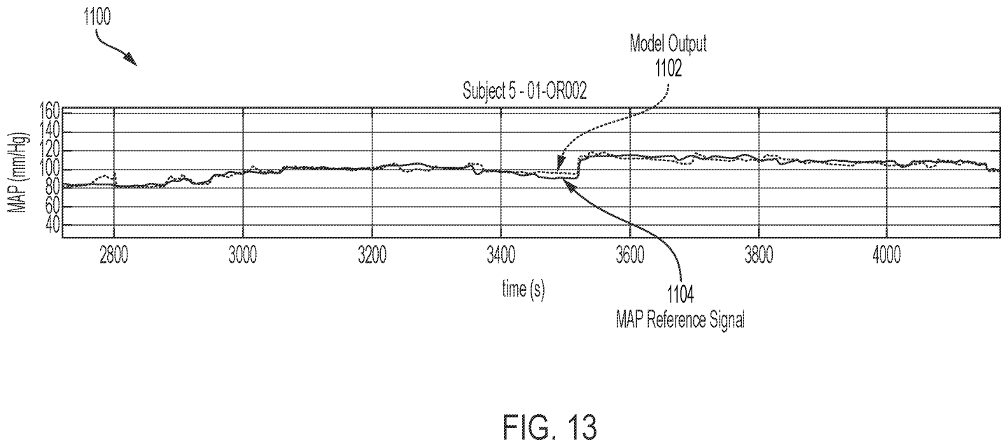

In additional exemplary embodiments, the deep learning model is a hybrid CNN-LSTM model, as is shown generally at 1000 in the flowchart of FIG. 12. In the flowchart of FIG. 12, a combined matrix 1002 is input into a CNN machine learning model at 1004, followed by LSTM machine learning model at 1006, with blood pressure output at 1008. As an example, FIG. 13 shows generally at 1100 an output 1102 from an LSTM model alongside a MAP reference signal 1104, where pulse features from a single probe location (e.g., finger) were fed in addition to the corresponding DPTT between two locations (e.g., ear-finger).

In additional exemplary embodiments, the CNIBP system and method may be at least periodically calibrated, e.g., to account for possible loss of accuracy over time, e.g., due to confounders affecting the model being used. In this case, the CNIBP system may be intermittently calibrated using a blood pressure cuff, such as cuff 11 in FIG. 1.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

The systems and methods described herein can be provided in the form of tangible and non-transitory machine-readable medium or media (such as a hard disk drive, hardware memory, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

From the foregoing, it will also be appreciated that various modifications can be made without deviating from the technology. For example, various components of the technology can be further divided into subcomponents, or various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A patient monitoring system, comprising:
a first PPG sensor in contact with a patient at a first location, the first sensor providing first data over a first time period related to the patient to determine one or more patient parameters;
a second PPG sensor in contact with a patient at a second patient location different from the first location, the second sensor providing second data over said first time period related to the patient to determine one or more patient parameters; and
a processor configured to:
compare at least a portion of the first data and at least a portion of the second data in order to calculate a differential pulse transit time (DPTT) between the first and second locations; and
determine continuous non-invasive blood pressure (CNIBP) using the first data, the second data and the DPTT.

2. The patient monitoring system of claim 1, wherein the processor is configured to compare at least one fiducial point in the first data and in the second data in order to calculate DPTT.

3. The patient monitoring system of claim 2, wherein the at least one fiducial point comprises a peak of the pulse, the trough of the pulse or the location of maximum upslope gradient.

4. The patient monitoring system of claim 1, wherein the processor is configured to input at least a portion of the first data, at least a portion of the second data, and the calculated DPTT into a deep learning AI model to determine CNIBP.

5. The patient monitoring system of claim 4, wherein the first data input comprises red and infrared PPG data from the first PPG sensor, and wherein the second data input comprises red and infrared PPG data from the second PPG sensor.

6. The patient monitoring system of claim 4, wherein the first data input comprises one or more features derived from the raw PPG signal from the first PPG sensor, and wherein the second data input comprises one or more corresponding features derived from the raw PPG signal from the second PPG sensor.

7. The patient monitoring system of claim 6, wherein the one or more features comprises one or more of: pulse duration, relative position of maximum upslope of the systolic rise, peak location and amplitude, perfusion index, baseline trend, respiratory cycle information, area of upstroke, downstroke, max gradient of upslope, and baseline value.

8. The patient monitoring system of claim 6, wherein said one or more features are calculated as a sequence of values over time.

9. The patient monitoring system of claim 4, wherein the deep learning AI model is an LSTM model, a CNN model, or a hybrid CNN-LSTM model.

10. The patient monitoring system of claim 1, further comprising a blood pressure cuff configured to intermittently calibrate the patient monitoring system.

11. A method for patient monitoring comprising:
configuring a first PPG sensor to contact a patient at a first location, the first sensor configured to provide first data over a first time period related to the patient to determine one or more patient parameters;
configuring a second PPG sensor to contact a patient at a second patient location different from the first location, the second sensor providing second data over said first time period related to the patient to determine one or more patient parameters; and
with a processor:
comparing at least a portion of the first data and at least a portion of the second data in order to calculate a differential pulse transit time (DPTT) between the first and second locations; and
determining continuous non-invasive blood pressure (CNIBP) using the first data, the second data and the DPTT.

12. The patient monitoring method of claim 11, wherein the processor is compares at least one fiducial point in the first data and in the second data in order to calculate DPTT.

13. The patient monitoring method of claim 12, wherein the at least one fiducial point comprises a peak of the pulse, the trough of the pulse or the location of maximum upslope gradient.

14. The patient monitoring method of claim 11, wherein the processor is configured to input at least a portion of the first data, at least a portion of the second data, and the calculated DPTT into a deep learning AI model to determine CNIBP.

15. The patient monitoring method of claim 14, wherein the first data input comprises red and infrared PPG data from the first PPG sensor, and wherein the second data input comprises red and infrared PPG data from the second PPG sensor.

16. The patient monitoring method of claim 14, wherein the first data input comprises one or more features derived from the raw PPG signal from the first PPG sensor, and wherein the second data input comprises one or more corresponding features derived from the raw PPG signal from the second PPG sensor.

17. The patient monitoring method of claim 16, wherein the one or more features comprises one or more of: pulse duration, relative position of maximum upslope of the systolic rise, peak location and amplitude, perfusion index, baseline trend, respiratory cycle information, area of upstroke, downstroke, max gradient of upslope, and baseline value.

18. The patient monitoring method of claim 16, wherein said one or more features are calculated as a sequence of values over time.

19. The patient monitoring system of claim 14, wherein the deep learning AI model is an LSTM model, a CNN model, or a hybrid CNN-LSTM model.

20. The patient monitoring method of claim 11, further comprising intermittently calibrating the patient monitoring system using a blood pressure cuff
